(19) United States
(12) Patent Application Publication
(10) Pub. No.: US 2007/0293817 A1
(43) Pub. Date: Dec. 20, 2007

(54) Portable IV infusion mornitoring system

(76) Inventors: Jun Feng, Cambridge, MA (US); Xueming Miao, Shanghai (CN)

Correspondence Address: Jun Feng, 150 Allston St., Cambridge, MA, 02139, US

(21) Appl. No.: 11/811,466

(22) Filed: Jun. 10, 2007

Related U.S. Application Data
(60) Provisional application No. 60/814,238, filed on Jun. 16, 2006.
(60) Provisional application No. 60/815,204, filed on Jun. 20, 2006.

(51) Int. Cl. A61M 31/00

(52) U.S. Cl. 604/65

(57) ABSTRACT

A portable infusion monitoring system displays the liquid level and flow rate data during infusion process, as well as gives an alarm for patents and nurses in hospital as the medical liquid in an IV bottle drops to a predetermined low level. This system comprises a set of liquid level sensor, a microprocessor, and a monitor terminal. The liquid level sensor generates an electric signal related to the liquid level inside the IV bottle. The microprocessor analyzes the electric parameters detected from the electric signal, and obtains the liquid level data. The liquid level data are sent to the monitor terminal for display, and an alarm is activated when the medical liquid inside the IV bottle drops to a predetermined low lever. Further functions of the monitor terminal includes an automatic switch to cut off the IV feeding process and send the alarm signal to a nurse station through signal network by wire or wirelessly. Several interference filtering methods are applied to increase signal/noise ratio, and therefore warrant the operation reliability.

### CROSS REFERENCE TO RELATED APPLICATIONS

This application claims the benefit of provisional patents: application No. 60/814,238, filed on Jun. 16, 2006, and application No. 60/815,204 filed on Jun. 20, 2006, by the present inventors to US Patent and Trademark Office.

### REFERENCES CITED

### U.S. Patent Documents

| | | | |
|---|---|---|---|
| 3,375,716 | Apr. 2, 1968 | Hersch | 73/304 |
| 3,390,577 | Jul. 2, 1968 | Phelps et al. | 73/194 |
| 3,450,153 | Jul. 17, 1969 | Hildebrandt et al. | 137/486 |
| 3,641,543 | Feb. 8, 1972 | Rigby | 73/861.41 |
| 3,939,360 | Feb. 17, 1976 | Jackson | 307/118 |
| 4,002,996 | Jan. 11, 1977 | Klebanoff et al. | 331/65 |
| 4,671,110 | Jun. 9, 1987 | De Kock | 73/323 |
| 4,749,988 | Jun. 7, 1988 | Berman et al. | 340/618 |
| 5,563,584 | Oct. 8, 1996 | Rader et al. | 340/618 |
| 6,964,278 | Nov. 15, 2005 | Tschanz | 137/392 |

### FEDERALLY SPONSORED RESEARCH

Not Applicable

### SEQUENCE LIST OR PROGRAM

Not Applicable

### FIELD OF INVENTION

The present invention relates to a portable monitoring system of the liquid level in a liquid feeding line, and more particularly to a portable IV infusion monitoring system.

### BACKGROUND OF THE INVENTION

Assume a patient lies on bed to receive IV infusion. There are two types of infusion systems. One is by pump, another is by gravity. The pump infusion system is very costly and often encounters maintenance trouble. Therefore, many hospital workers prefer to use the traditional gravity infusion system. The gravity IV infusion line consists of three parts: a) An IV bottle contains medical liquid and air above the medical liquid; (b) Infusion line includes a liquid needle inserted inside the IV bottle to receive medical liquid, a plastic tube (liquid tube) with one end connected to the liquid needle as liquid inlet and another end connected to the IV injection needle for injecting the medical liquid into the patient vein. A flow rate switch is located in the middle of the plastic tube to control the flow rate manually; (c) Air line includes an air needle inserted into the IV bottle to apply air pressure for driving the liquid flow, and a plastic tube (air tube) with one end connected to the air tube as air outlet and another end opened to the environment as air inlet. As the medical liquid in the IV bottle drops to a predetermined low level, i.e., nearly finished, the bottle must be replaced by a new one, otherwise air may enter the infusion line and causes serious medical problems.

So far, the job of bottle replacement needs frequent supervision from patient and nurses by eyeball. This task becomes a heavy burden of medical workers, particularly at night. To develop an alarming system for IV infusion becomes a big demand from hospitals and patients. Many efforts have been done in this field.

U.S. Pat. No. 3,375,716 to Hersch discloses a fluid quantity measuring device including a sensing capacitor to measure the prevailing quantity of fluid in a container. Hersch's disclosure uses a time-constant circuit, and therefore the measurement accuracy is very poor as well the poor reliability, both of which are very critical in medical application. The present invention applies a microprocessor including an interference filtering means, which acts as a mini computer to process all received electric parameters from the electrodes in digital format, and therefore warrant a very high accuracy and high reliability. Further more, the present invention uses sound alarm, terminal display and signal network to further ease the hospital works.

U.S. Pat. No. 3,390,577 to Phelps et al. discloses a monitoring system for fluid flow in drop form. Phelps' disclosure only applies for measuring the liquid drop. Such system is poor in accuracy and reliability. The present invention can measure the liquid level data at any time moment. Further more, the present invention applies a microprocessor, which acts as a mini computer to process all received electric parameters from the electrodes in digital format, and therefore warrant a very high accuracy and high reliability. In addition, the present invention uses sound alarm, terminal display and signal network to further ease the hospital works.

U.S. Pat. No. 3,641,543 to Rigby discloses a low-level detector and drop rate monitor that can only detect the low solution level and drop rate. Rigby's first embodiment is for detecting a low solution level, where the conductor means needs to be mounted on opposite one another in juxtaposition, one of the conductors and a multivibrator means are required to be grounded. His second embodiment is for monitoring drop rate, where the two electrode means must be placed diametrically opposite one another in juxtaposition, a stabilizing means and a tachometer means are required. The present invention can detect all liquid infusion information including the liquid level at any time moment and liquid moving rate including the low liquid level. Furthermore the present invention does not require two electrodes being placed on opposite one another, does not require any element to be grounded and therefore is portable. In addition, the present invention does not need stabilizing means and tachometer means for operation.

U.S. Pat. No. 3,939,360 to Jackson discloses a liquid level sensor and electrode assembly therefore. Jackson's disclosure requires three capacitance plates to measure the capacitance. Furthermore, the circuit means uses analog signal for measurement, and therefore results in a poor accuracy and poor reliability. The present invention needs only minimum two electrodes for measurement, and the signal process is accomplished by a microprocessor, therefore, warrant a high accuracy and high reliability.

U.S. Pat. No. 4,002,996 to Klebanoff et al. discloses a level detector using oscillator circuit with two capacitive probes. Klebanoff's disclosure detects the low liquid level by emitting an oscillation using a feed-back network. The present invention applies the received signal to microprocessor and send out an alarm signal when the microprocessor analyzes the digital data and finds that the liquid level has dropped to a predetermined low level.

U.S. Pat. No. 4,671,110 to de Kock discloses a level sensing device. De Kock's disclosure is for sensing the liquid level in a boiler or vessel, and therefore need to have tublar glass and a conduit for communication with the liquid inside the vessel. One of the conductors needs to contact the liquid inside the vessel. The present invention is for detecting the liquid level in an IV bottle, and does not need any contact with the liquid inside the liquid container.

U.S. Pat. No. 4,749,988 to Berman et al. discloses a non-invasive liquid level sensor. Berman's disclosure requires the outer shield conductor of a shielded cable to be grounded in order to avoid external interference to the electrode, and therefore such a sensor is not portable. Furthermore, his disclosure does not include any signal process element and signal terminal equipment. The present patent does not need any part to be grounded and therefore is portable. The present invention includes a microprocessor acting as a mini computer, and all the interferences from environment are processed in the microprocessor to be filtered out. Furthermore, the present invention includes the monitor terminal for alarm and display.

U.S. Pat. No. 5,563,584 to Rader et al. discloses a liquid level sensing and monitoring system for medical fluid infusion systems. Rader's disclosure applies pressure sensor technology. In his second embodiment, a sensor is inserted into the outlet of a liquid container and contacts the liquid for detecting the liquid level. The present invention applied the impedance sensor, and none of elements in the present invention needs to be inserted into the outlet of a liquid container.

U.S. Pat. No. 6,964,278 to Tschanz discloses a non-invasive gauge glass liquid level sensor apparatus. Tschanz's disclosure is for sensing liquid level in a boiler or other vessel. Therefore his apparatus requires a tubular gauge glass. In addition, the boiler or vessel must be metallic material. The present invention is for monitoring IV infusion liquid level, and does not requires a gauge glass as well as a metallic material for the liquid container.

The present invention provides a portable IV infusion monitoring system, which is capable for displaying the liquid level and flow rate, as well as giving alarm when the medical liquid in the bottle drops to a predetermined low level. The present invention is different from and superior over all the pri-arts in structure, cost, accuracy and reliability, as well as in ease of use.

### SUMMARY OF THE INVENTION

A portable IV infusion monitoring system is provided to display the liquid level data and to give alarm as the medical liquid in the IV bottle drops to a predetermined low level. The IV infusion is used for injecting a medical liquid to a patient vein. It includes an IV bottle containing medical liquid and air above the medical liquid. Both a liquid needle for liquid flow and an air needle for air flow are inserted into the IV bottle. A plastic liquid tube for liquid flow is connected at the end of the liquid needle. A plastic air tube for air flow is connected at the end of the air needle.

The first embodiment of the present invention comprises a set of liquid level sensor including at least two electrodes, a microprocessor, and a monitor terminal. The power is provided preferably by a battery or an external power source as an option to user. The at least two electrodes are located at either two sides of the IV bottle in opposite direction or one side of the IV bottle in parallel location. They are capable of conducting an electric current between them, e.g., an alternating current. The microprocessor acting as a mini computer is capable of detecting the electric parameters of the alternating current, analyzing the electric parameters to obtain the liquid level data inside the IV bottle, and sending all the liquid level data to the monitor terminal. The electric parameters related to the liquid level include at least one of voltage, current, impedance, phase and frequency etc. The liquid level data include the liquid level inside the IV bottle at any time moment, the liquid flow rate during infusion process, and the comparison with the predetermined low level. The monitor terminal includes an alarm means for sending an alarm, and a display means to display the liquid level data in a terminal screen. Alternatively, the liquid level sensor uses an electric bridge to detect the electric signal for better accuracy.

The microprocessor acting as a mini computer includes a control means for applying the alternating current to the at least two electrodes, receiver means for receiving the electric signal of the alternating current, detector means for detecting the electric parameters of the electric signal, process means for analyzing the electric parameters and obtaining the liquid level data inside the IV bottle, and transmission means for sending out the liquid level data. Each of above elements may be built together in one chip, or they can stand alone as individual circuit or chip. The control means includes at least one of an oscillator, an oscillator circuit, a logic circuit etc. The receiver means includes at least one of an input pot, an amplifier, a filter etc. The detector means includes at least one of a C/V converter (capacitance to voltage converter), a differential circuit, or a voltage meter etc. The process means includes at least one of signal interface, A/D converter, digital register, processor, or logic circuit etc. The transmission means includes at least one of output pot, conductive wire or antenna etc.

Alternatively the microprocessor includes receiver for receiving the electric signal of the alternating current, detector for detecting the voltage signal from the electric signal, signal interface for storing the voltage signal, A/D converter for converting the voltage signal into digital data, digital register for storing the digital data, processor for analyzing the digital data and obtaining the liquid level data, output port for transmitting the liquid level data. All the functions of each element are controlled by program controller, which is programmed with unique software code for administrating the operation of all above elements. Each of above elements may be built together in one chip, or they can stand alone as individual circuit or chip. The microprocessor further includes an interference filtering means for removing all interference from the environment.

The electric interference from environment often degrades or sometime disables the normal operation of such a monitoring system. Therefore, to move the signal interference becomes very critical in order to obtain high accuracy and high reliability of the monitoring work. In a typical electric environment, at least one shielding plate made of conductive materials is placed on the outer surface of each electrode. The shielding plate is insulated to the electrodes. The at least one shielding plate is connected to a reference point with zero potential, e.g., the negative pole of a battery. Alternatively, the interference signal in the at least one shielding plate is passed over to the microprocessor, and it is then filtered out in signal processing. Meanwhile, at least two coaxial cables consist of a center conductor surrounded by a concentric outer shielding layer made of conductive materials. The center conductor is insulated from the outer shielding layer. The center conductors of the at least two coaxial cables connect the at least two electrodes to the microprocessor for transmitting the signal. The outer shielding layers of the at least two coaxial cables are connected to the reference point with zero potential, e.g., the negative pole of a battery. Alternatively, the outer shielding layers are connected to the microprocessor for interference filtering process. The microprocessor, part of the monitor terminal and the battery are contained in an assembly box. To shielding the microprocessor and other parts inside the assembly box from the environmental interference, either the assembly box is made of metal or the assembly box is coated with conductive materials. The coated methods include chemical coating, physical coating, mechanical coating, or a simple metal lining. Similar to the shielding plate, the conductive part of the assembly box is connected to a reference point with zero potential, e.g., the negative pole of the battery. Alternatively, the environmental noise in the assembly box is passed over to the microprocessor for interference filtering.

However, if the electric environment is very noisy, and the interference becomes too strong to perform a normal operation of this monitoring system, the signal interference from the environment can be removed by special signal processing methods. The control means in the microprocessor generates the alternating current in various forms such as narrow band signal, multi-frequency signal, and encoded signal (containing continuous wave, pulse and digital signal etc.). If a narrow band signal is applied, the interference filtering means in the microprocessor has a narrow band filter, which can filter out the signal within this narrow band, and remove all random interference outside the narrow band. If a multi-frequency signal is applied, the interference filtering means has a Fourier analyzer, which can perform Fourier analysis to pick up the right signal, and remove the noise interference. If an encode signal is applied, the interference filtering means has a decoder, which can perform decoding to pick up the right signal, and remove the noise interference. The way of encoding includes frequency modulation, angle modulation, phase modulation, pulse modulation, pulse code modulation, FDMA and CDMA modulations etc. All above filtering methods are more effective in digital format

Further alternatively, two pairs of electrodes can be positioned in parallel outside the IV bottle 11. By differentiation of the signal or electric parameters, the environmental interference will be removed too. Hereby there is no need of grounding in order to avoid the environmental interference since this monitoring system is designed as a portable device.

In addition to the environmental interference, the signal deformation may also reduce the reliability of the monitoring system, e.g., in the case of flexible IV bag (i.e., a soft IV bottle), the bag may deform during infusion process and therefore lead to the deformation of the electrical signal and related electrical parameters. However, such signal deformation can be analyzed by the microprocessor, and the corrected electric parameters can be picked up by the analysis. Therefore, it would be impossible to obtain high accuracy and high reliability without the microprocessor.

The monitor terminal includes alarm means for providing an alarm, and display means for displaying the liquid level data in a terminal screen. The alarm means includes a sound generator for giving a loud sound when the medical liquid level inside the IV bottle drops below the predetermined low level. Alternatively the alarm means includes a switch means for cutting off the feeding of medical liquid when the medical liquid level inside the IV bottle drops below the predetermined low level. Further alternatively, the alarm means includes a signal network for sending the liquid level data by the network to a nurse station through wire or wirelessly.

The monitor terminal further includes a rate controller for controlling the infusion rate according to a predetermined rate value. The rate controller comprises an input port for inputting the desired infusion rate of the medical liquid inside the IV bottle, a comparator for comparing the desired infusion rate and the detected infusion rate, and an electric switch means for adjusting the infusion rate according to the results from the comparator.

The second embodiment is similar to the first embodiment, but only at least one electrode is positioned outside the IV bottle. Meanwhile, a conductive wire is connected to either the liquid needle or the air needle inside the IV bottle. This embodiment is especially usable for an old IV infusion system, where both the liquid needle and air needle are made of metal. When an electric current (e.g., an alternating current) is applied between the electrode and the conductive wire, the electric parameters (e.g., impedance) between the liquid and the electrode are detected by the detector means in microprocessor since the alternating current goes through the conductive wire to the metallic needle to the liquid and finally to the electrode. Again the environmental interference is a critical issue for operation reliability. The methods and devices for removing the interference are similar to those in the first embodiment.

The third embodiment of the present invention includes a set of liquid level sensor comprising a first conductive wire connected to the air needle and a second conductive wire connected to the liquid needle, a signal processor, and an alarm device. All the monitoring system is powered preferably by a battery or an external source as an option to user. The signal processor comprising electronic circuits is capable of applying an electric current, (preferably direct current, but alternating current as an option) between the two conductive wires, detecting the electric parameters of the electric current, analyzing the electric parameters related to the liquid level inside the IV bottle, and sending an alarm signal when the medical liquid in the IV bottle drops to a predetermined low level. As the electric current is applied between the two conductive wires, the electric parameters (e.g., impedance) between the two needles are detected. The alarm device is capable of giving alarm to patient and nurses after receiving the alarm signal from the signal processor. The interference removing methods are similar to those in the first and the second embodiment.

The signal processor in the third embodiment comprises control means for applying an electric current between the two conductive wires, receiver means for receiving the electric parameters of the electric current, process means for analyzing the received electric parameter and judging if the medical liquid inside the IV bottle has dropped below the predetermined low level, transmission means for sending an alarm signal if the liquid level has dropped to the predetermined low level. Alternatively, an electric bridge is used in the liquid level sensor to increase detection accuracy.

### BRIEF DESCRIPTION OF THE DRAWINGS

FIG. 1 is a schematic drawing of the first embodiment of a portable IV infusion monitoring system for the present invention.

FIG. 2 is the schematic drawing of an alternative first embodiment of the present invention.

FIG. 3 is a schematic drawing of an alternative liquid level sensor for the present invention.

FIG. 3A is a schematic drawing of an exemplary coaxial cable, shielding plate and assembly box for removing the environmental interference in the present invention.

FIG. 4 is a block diagram of an exemplary microprocessor for the present invention.

FIG. 4A is a block diagram of an alternative microprocessor for the present invention.

FIG. 4B is a block diagram of three alternative embodiments of the interference filtering means in the microprocessor for the present invention.

FIG. 5 is a block diagram of an exemplary monitor terminal for the present invention.

FIG. 5A-5C are block diagrams of exemplary and alternative alarm means for the present invention.

FIG. 5D is a block diagram of an alternative monitor terminal for the present invention.

FIG. 6 is a schematic drawing of the second embodiment of a portable IV infusion monitoring system for the present invention.

FIG. 7 is a schematic drawing of the third embodiment of a portable IV infusion monitoring for the present invention.

FIG. 8 is a block diagram of an exemplary signal processor for the third embodiment of the present invention.

FIG. 9 is a block diagram of an exemplary alarm device for the third embodiment of the present invention.

### DETAILED DESCRIPTION OF THE ILLUSTRATIVE EMBODIMENTS

In describing preferred embodiment of the present invention illustrated in the drawings, specific terminology is employed for the sake of clarity. However, the invention is not intended to be limited to the specific terminology so selected, and it is to be understood that each specific element includes all technical equivalents which operate in a similar manner to accomplish a similar purpose.

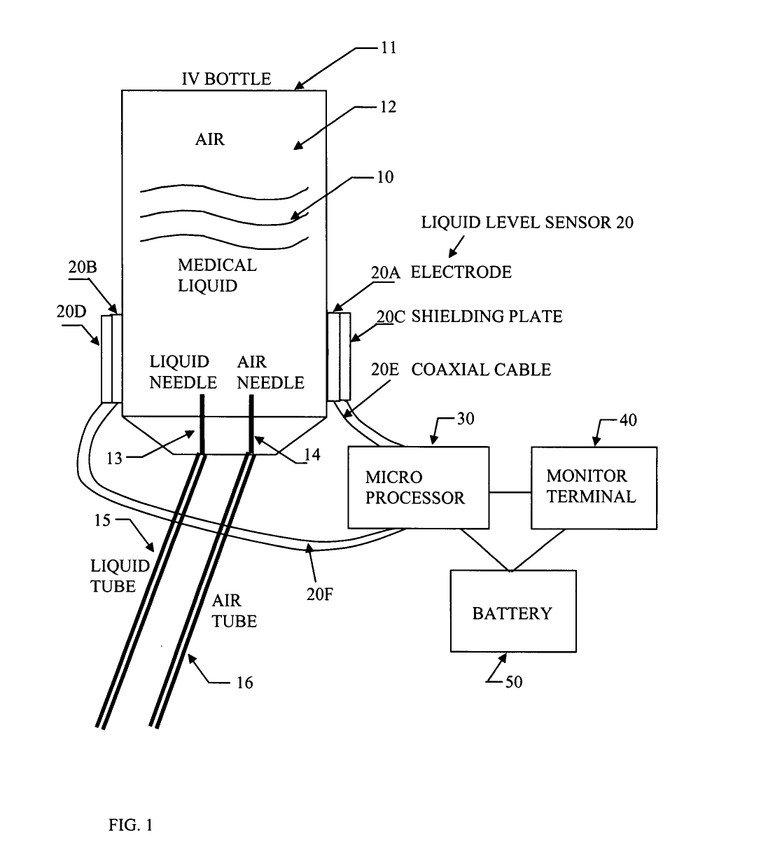

FIG. 1 is a schematic drawing of the first embodiment of a portable IV infusion monitoring system that is capable of detecting the liquid level of the medical liquid 10 inside an IV bottle 11, and giving alarm when the medical liquid 10 in the IV bottle 11 drops to a predetermined low level.

The IV infusion system comprises the IV bottle 11 containing the medical liquid 10 and air 12 above the medical liquid 10. A liquid needle 13 and an air needle 14 are inserted into the IV bottle 11. A liquid tube 15 is connected at the end of the liquid needle 13. An air tube 16 is connected at the end of the air needle 14. The IV bottle 11 can be made of stiff materials such as glass or harden plastics, or it can be made of flexible plastic bags.

The IV infusion monitoring system comprises a liquid level sensor 20 including at least two electrodes 20A, 20B, a microprocessor 30, and a monitor terminal 40. The power is provided preferably by a battery 50 or by an external power source as an option to user. The at least two electrodes 20A, 20B are located at two sides of the IV bottle 11 in opposite direction with each other, and are capable of conducting an alternating current between them. The microprocessor 30 acting as a mini computer is capable of detecting the electric parameters of the alternating current, analyzing the electric parameters to obtain the liquid level data inside the IV bottle 11, and sending all the liquid level data to the monitor terminal 40. The electric parameters related to the liquid level include at least one of voltage, current, impedance, phase and frequency etc. The liquid level data includes the liquid level inside the IV bottle 11 at any time moment, the liquid flow rate during infusion process, and the comparison with the predetermined low level. The monitor terminal 40 includes an alarm means for sending out an alarm signal to activate an alarm to patient and nurses if the medical liquid 10 has dropped to the predetermined low level.

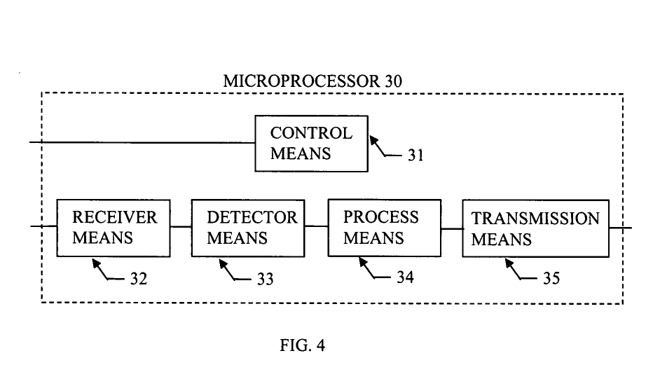

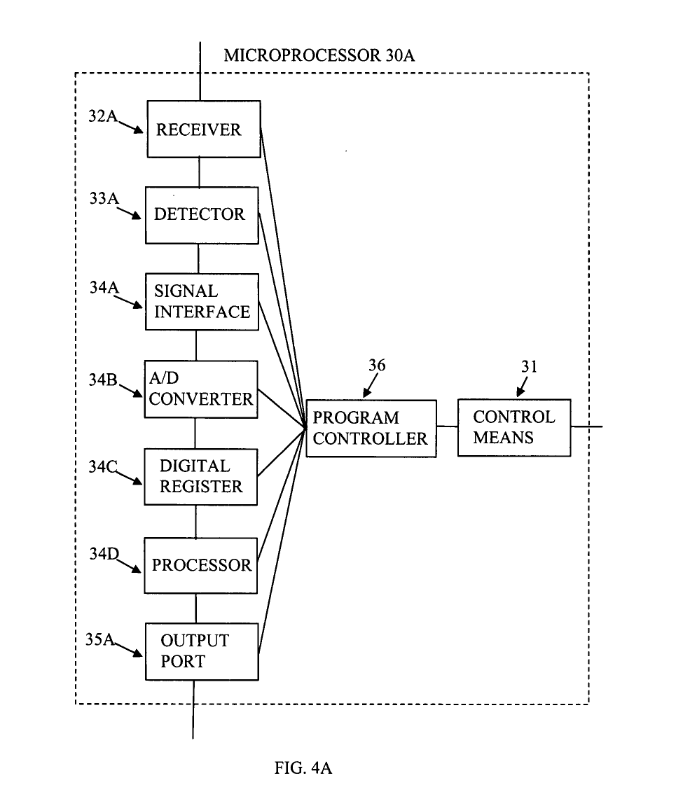

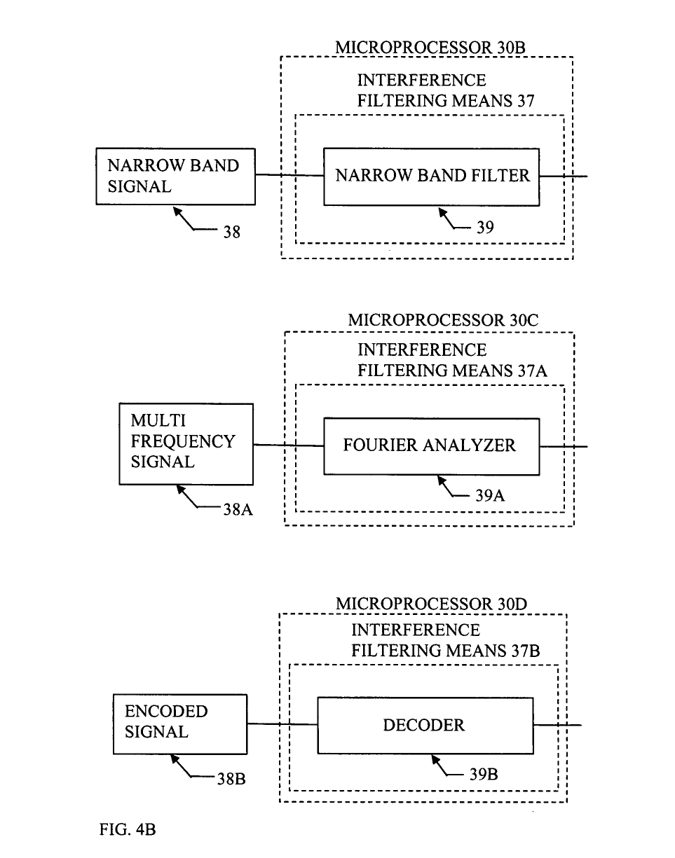

In a typical electric environment, at least one shielding plate 20C, 20D made of conductive materials is placed on the outer surface of each electrode 20A, 20B, and is insulated from the electrodes 20A, 20B. The at least one shielding plate 20C, 20D is connected to a reference point with zero potential, e.g., the negative pole of a battery 50. Alternatively, the interference signal in the at least one shielding plate is passed over to the microprocessor 30, and it is then filtered out in signal processing. Meanwhile, at least two coaxial cables 20E, 20F consist of a center conductor surrounded by a concentric outer shielding layer made of conductive materials. The center conductor is insulated with the outer shielding layer. The center conductors of the at least two coaxial cables 20E, 20F connect the at least two electrodes 20A, 20B to the microprocessor 30 for transmitting the signal. The outer shielding layers of the at least two coaxial cables 20E, 20F are connected to the reference point with zero potential, e.g., the negative pole of a battery 50. Alternatively, the outer shielding layers are connected to the microprocessor 30 for interference filtering process. However, if the electric environment is very noisy, and the interference becomes too strong to perform a normal operation of this monitoring system, the signal interference from the environment can be removed by special signal processing methods described in FIG. 4B. Further alternatively, two pairs of electrodes can be positioned in parallel outside the IV bottle 11. By differentiation of the signal or electric parameters, the environmental interference will be removed too. Hereby there is no need of grounding in order to avoid the environmental interference since this monitoring system is designed as a portable device.

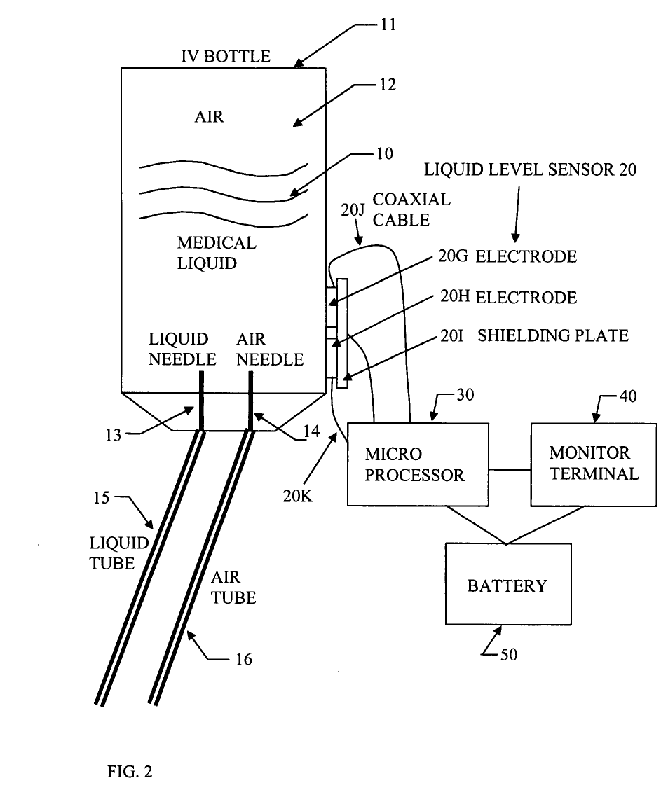

FIG. 2 is the schematic drawing of an alternative embodiment of the present invention. It is similar to the embodiment of FIG. 1, but the liquid level sensor 20 includes at least two electrodes 20G, 20H, which are positioned at one side of the IV bottle 11 in parallel location.

Again, for removing the interference from the environment, at least one shielding plate 201 made of conductive materials is placed on the outer surface of each electrode 20G, 20H. The at least one shielding plate 201 is connected to a reference point with zero potential, e.g., the negative pole of a battery 50. Alternatively, the interference signal in the at least one shielding plate 201 is passed over to the microprocessor 30, and it is then filtered out in signal processing. Meanwhile, at least two coaxial cables 20J, 20K consist of a center conductor surrounded by a concentric outer shielding layer made of conductive materials. The center conductors of the at least two coaxial cables 20J, 20K connect the at least two electrodes 20G, 20H to the microprocessor 30 for transmitting the signal. The outer shielding layers of the at least two coaxial cables 20J, 20K are connected to the reference point with zero potential, e.g., the negative pole of a battery 50. Alternatively, the outer shielding layers are connected to the microprocessor 30 for interference filtering process. However, if the electric environment is too noisy to perform the normal operation of this monitoring system, the signal processing methods described in FIG. 4B can be applied to remove most environmental interference. Further alternatively, two pairs of electrodes can be positioned in parallel outside the IV bottle. By differentiation of the signal or electric parameters, the environmental interference will be removed too.

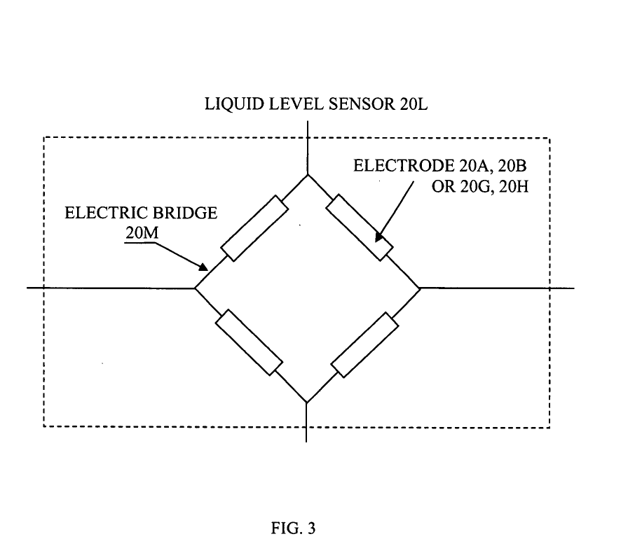

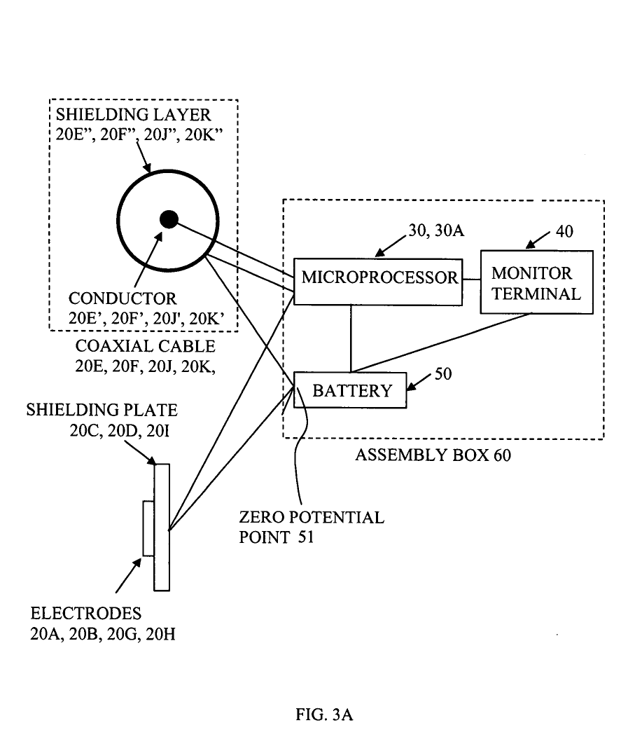

FIG. 3 is a schematic drawing of an alternative liquid level sensor 20L. The liquid level sensor 20L uses an electric bridge 20M to detect the electric signal of the alternating current. The electric bridge 20M contains the at least two electrodes 20A, 20B in FIG. 1, or 20G, 20H in FIG. 2.

FIG. 3A is a schematic drawing of an exemplary coaxial cable, shielding plate and assembly box for removing the environmental interference. The at least one shielding plate 20C, 20D, 201 is connected to a reference point with zero potential 51, e.g., the negative pole of a battery 50. The at least one shielding plate 20C, 20D, 201 is insulated from the electrodes 20A, 20B, 20G, 20H. Alternatively, the interference signal in the at least one shielding plate 20C, 20D, 201 is passed over to the microprocessor 30, 30A and it is then filtered out in signal processing. Meanwhile, at least two coaxial cables 20E, 20F, 20J, 20K consist of a center conductor 20E′, 20F′, 20J′, 20K′ surrounded by a concentric outer shielding layer 20E″, 20F″, 20J″, 20K″ made of conductive materials. The center conductors 20E′, 20F′, 20J′, 20K′ are insulated from the outer shielding layers 20E″, 20F″, 20J″, 20K″. The center conductors 20E′, 20F′, 20J′, 20K′ of the at least two coaxial cables 20E, 20F, 20J, 20K connect the at least two electrodes 20A, 20B, 20G, 20H to the microprocessor 30, 30A for transmitting the signal. The outer shielding layers 20E″, 20F″, 20J″, 20K″ of the at least two coaxial cables 20E, 20F, 20J, 20K are connected to the reference point with zero potential 51, e.g., the negative pole of a battery 50. Alternatively, the outer shielding layers 20E″, 20F″, 20J″, 20K″ are connected to the microprocessor 30, 30A for interference filtering process. The at least two coaxial cables 20E, 20F, 20J, 20K are in a form of wire, or string, or strip, or twisted-pair, or cable. The center conductor 20E′, 20F′, 20J′, 20K′ is made of solid conductor, e.g., copper in a form of single wire, stranded wires or twist-pair (i.e, two insulated strands of conductive wire twisted around each other). The outer shielding layer 20E″, 20F″, 20J″, 20K″ is made of at least one foil insulation and braided metal, for example, it could be dual shielding (i.e., one layer of foil insulation and one layer of braided metal shielding), or quad shielding (i.e., two layers of foil insulation and two layers of braided metal shielding) if the environmental interference is strong. The coaxial cable 20E, 20F, 20J, 20K has high resistance not only to noise interference, but also to attenuation. The microprocessor 30, 30A, part of the monitor terminal 40 and the battery 50 are contained in an assembly box 60. To shielding the microprocessor 30, 30A and other parts inside the assembly box 60 from the environmental interference, either the assembly box 60 is made of metal or the assembly box 60 is coated with conductive materials. The coated methods include chemical coating, physical coating, mechanical coating, or a simple metal lining etc. Similar to the shielding plate 20C, 20D, 201, the conductive part of the assembly box 60 is connected to a reference point with zero potential 51, e.g., the negative pole of the battery 50. Alternatively, the environmental noise in the assembly box 60 is passed over to the microprocessor 30, 30A for interference filtering.

FIG. 4 is a block diagram of an exemplary microprocessor 30. The microprocessor 30 acting as a mini computer includes a control means 31 for applying the alternating current to the at least two electrodes 20A, 20B in FIG. 1 and 20E, 20F in FIG. 2, receiver means 32 for receiving the electric signal of the alternating current, detector means 33 for detecting the electric parameters of the electric signal, process means 34 for analyzing the electric parameters and obtaining the liquid level data inside the IV bottle 11, and transmission means 35 for sending out the liquid level data. Each of above elements may be built together in one chip, or they can stand alone as individual circuit or chip. The control means 31 includes at least one of an oscillator, an oscillator circuit, a logic circuit etc. The receiver means 32 includes at least one of an input port, an amplifier, a filter etc. The detector means 33 includes at least one of a C/V converter (capacitance to voltage converter), a differential circuit, or a voltage meter etc. The process means 34 includes at least one of signal interface, A/D converter, digital register, processor, or logic circuit etc. The transmission means 35 includes at least one of output port, conductive wire or antenna etc.

FIG. 4A is a block diagram of an alternative microprocessor 30A. The microprocessor 30A comprises receiver 32A for receiving the electric signal of the alternating current, detector 33A for detecting the voltage signal from the electric signal, signal interface 34A for storing the voltage signal, A/D converter 34B for converting the voltage signal into digital data, digital register 34C for storing the digital data, processor 34D for analyzing the digital data and obtaining the liquid level data, output port 35A for transmitting the liquid level data. All the functions of each element are controlled by program controller 36, which is programmed with unique software code for administrating the operation of all above elements. Each of above elements may be built together in one chip, or they can stand alone as individual circuit or chip.

FIG. 4B is a block diagram of three alternative embodiments of the interference filtering means 37, 37A, 37B, which are included in microprocessor 30B, 30C, 30D respectively. The control means 31 in microprocessor 30, 30A of FIGS. 4 and 4A generates the alternating current, which is in various forms including narrow band signal 38, multi-frequency signal 38A or an encode signal 38B. These signals are passed over to the liquid level sensor 20, and then received by microprocessor 30, 30A, 30B, 30C, 30D. For a narrow band signal 38, the interference filtering means 37 includes a narrow band filter 39, which can filter out the signal within this narrow band, and removing all random interference outside the narrow band. For a multi-frequency signal 38A, the interference filtering means 37A includes a Fourier analyzer 39A, which can perform Fourier analysis to pick up the right signal, and remove the noise interference. For an encode signal 38B, the interference filtering means 37B includes a decoder 39B, which can perform decoding to pick up the right signal, and remove the noise interference. The form of signal includes single frequency signal, continuous wave, pulse signal, impulse signal, digital signal, spread spectrum signal and encoded signal etc. The way of encoding includes frequency modulation, angle modulation, phase modulation, pulse modulation, pulse code modulation, FDMA and CDMA modulations etc. All the above interference filtering methods are more effective in digital format.

In addition to the environmental interference, the signal deformation may also reduce the reliability of the monitoring system, e.g., in the case of flexible IV bag, the bag may deform during infusion process and therefore lead to the deformation of the electrical signal and related electrical parameters. However, such signal deformation can be analyzed by the microprocessor 30, and the corrected electric parameters can be picked up by the analysis. Therefore, it would be impossible to obtain high accuracy and high reliability without the microprocessor 30.

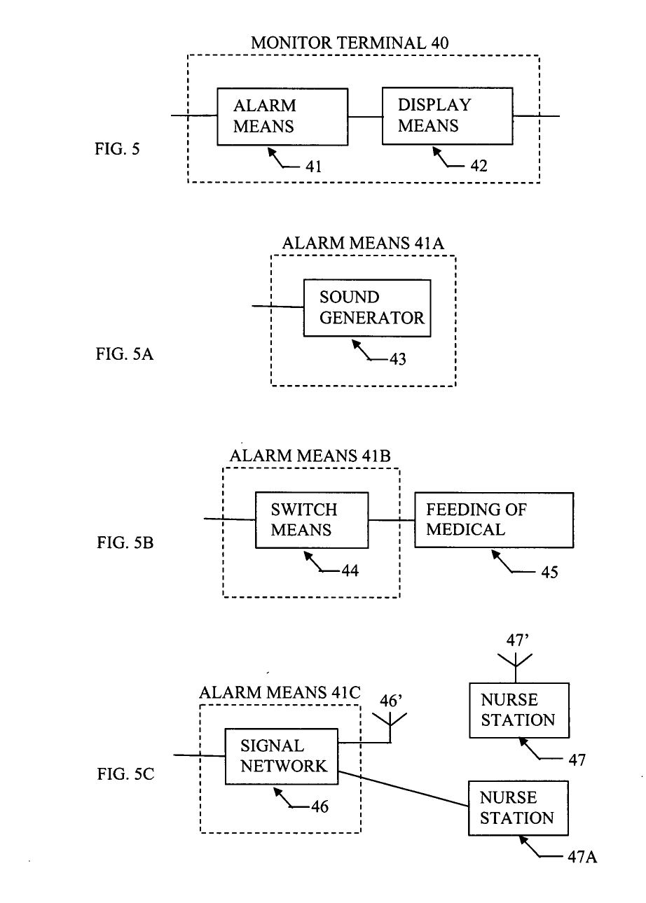

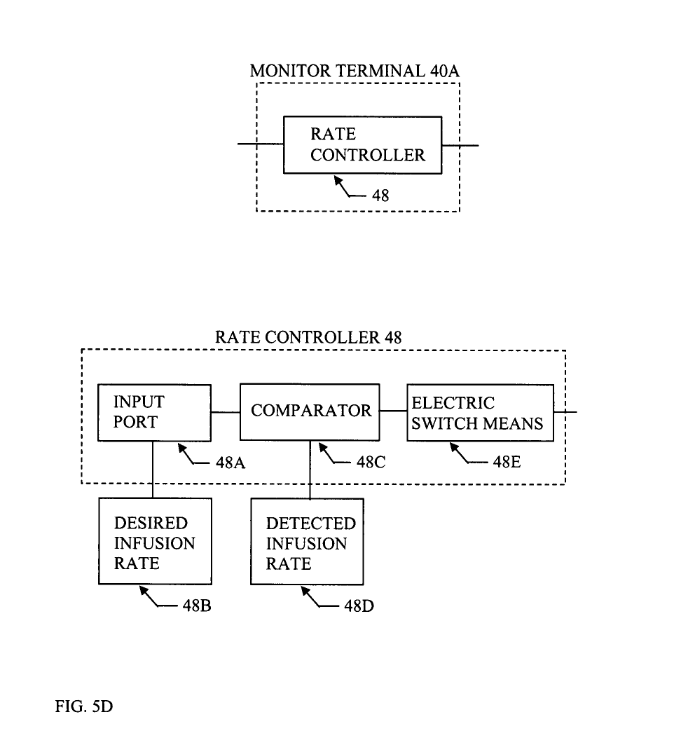

FIG. 5 is a block diagram of an exemplary monitor terminal 40. The monitor terminal 40 includes alarm means 41 for providing an alarm, and display means 42 for displaying the liquid level data in a terminal screen.

FIG. 5A is a block diagram of an exemplary alarm means 41A. The alarm means 41A includes a sound generator 43 for giving a loud sound when the medical liquid level inside the IV bottle 11 in FIGS. 1, 2 drops to the predetermined low level.

FIG. 5B is a block diagram of an alternative alarm means 41B. The alarm means 41B includes a switch means 44 for cutting off the feeding of medical liquid 45 when the medical liquid level inside the IV bottle 11 in FIGS. 1, 2 drops to the predetermined low level.

FIG. 5C is a block diagram of another alternative alarm means 41C. The alarm means 41C including a signal network 46 for sending the liquid level data by the signal network 46 to either a nurse station 47 wirelessly from an antenna 46′ to an antenna 47″, or a nurse station 47A by wire.

FIG. 5D is a block diagram of an alternative monitor terminal 40A. The monitor terminal 40A comprises a rate controller 48 for controlling the infusion rate according to a predetermined rate value. The rate controller 48 includes an input port 48A for inputting the desired infusion rate 48B of the medical liquid 10 inside the IV bottle 11, a comparator 48C for comparing the desired infusion rate 48B and the detected infusion rate 48D, and an electric switch means 48E for adjusting the infusion rate according to the results from the comparator.

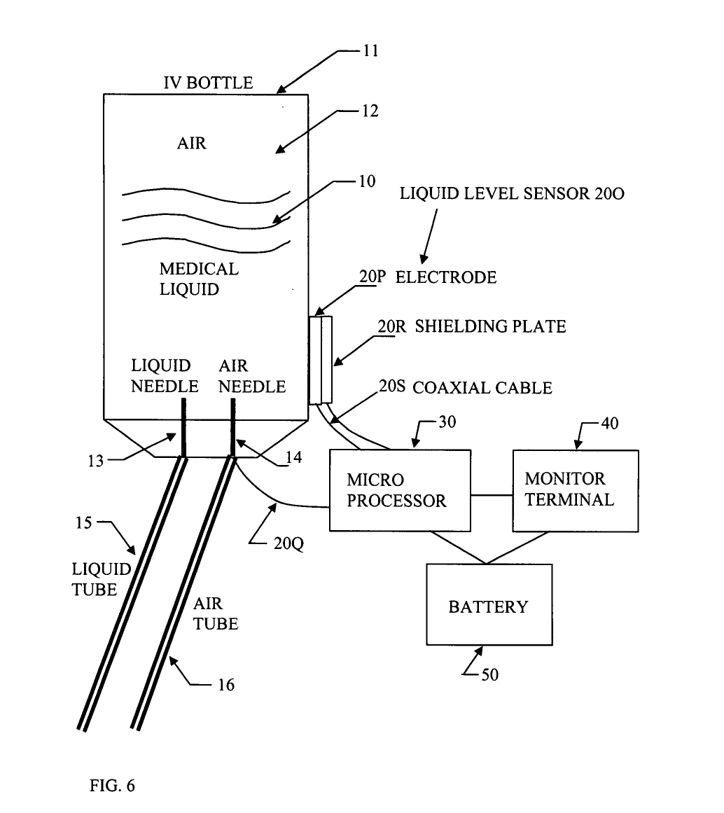

FIG. 6 is a schematic drawing of the second embodiment of a portable IV infusion monitoring system that is capable of detecting the liquid level of the medical liquid 10 inside an IV bottle 11, and giving alarm when the medical liquid 10 in the IV bottle 11 drops to a predetermined low level.

The second embodiment is similar to the first embodiment in FIG. 1, but only at least one electrode 20P is positioned outside the IV bottle 11. Meanwhile, a conductive wire 20Q is connected to either the liquid needle 13 or the air needle 14. This embodiment is especially usable for an old IV infusion system, where both the liquid needle 13 and air needle 14 are made of metal. When an alternating current is applied between the electrode 20P and the conductive wire 20Q, the electric parameters (e.g., impedance) between the medical liquid 10 and the electrode 20P is detected since the alternating current goes through the conductive wire 20Q to the metallic needle 13 or 14 to the medical liquid 10 and finally to the electrode 20P.

For removing the environmental interference, similar to the first embodiment, at least one shielding plate 20R made of conductive materials is placed on the outer surface of the electrode 20P. Meanwhile, at least one coaxial cable 20S connects the at least one electrode 20P to the microprocessor 30 for transmitting the signal, and the conductive wire 20Q is made of coaxial cable. The outer shielding layers of the coaxial cable 20Q, 20S are connected to the battery 50 or/and the microprocessor 30. The assembly box 60 in FIG. 3A containing the microprocessor 30, the battery 50 and part of the monitor terminal 40 also provides shielding function. However, if the electric environment is too noisy to perform the normal operation of this monitoring system, the signal processing described in FIG. 4B is performed to remove most environmental interference.

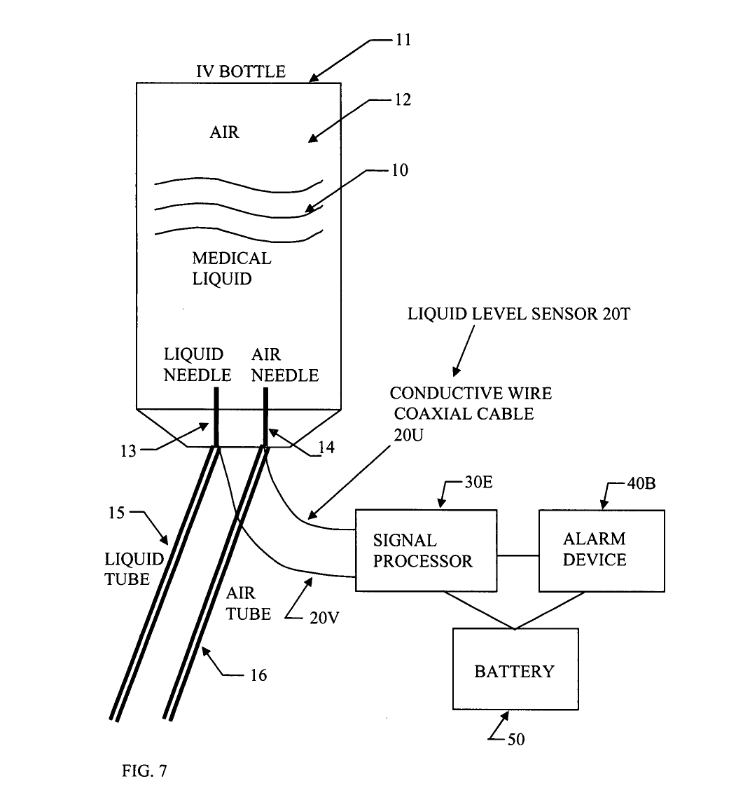

FIG. 7 is a schematic drawing of the third embodiment of a portable IV infusion monitoring system that is capable of giving alarm when the medical liquid 10 in the IV bottle 11 drops to a predetermined low level.

The third embodiment of the present invention comprises a liquid level sensor 20T including a first conductive wire 20U connected to the air needle 14, a second conductive wire 20V connected to the liquid needle 13, a signal processor 30E, and an alarm device 40B. All the monitoring system is powered preferably by a battery 50 or an external source as an option to user. The signal processor 30E comprising electronic circuits is capable of applying an electric current (preferably a direct current, but an alternating current as an option) between the two conductive wires 20U and 20V, detecting the electric parameters of the electric current, analyzing the electric parameters related to the liquid level inside the IV bottle 11, and sending an alarm signal when the medical liquid 10 in the IV bottle 11 drops to a predetermined low level. As the electric current is applied between the two conductive wires 20U and 20V, the electric parameters (e.g., impedance) between the two needles 13 and 14 are detected. The alarm device 40B is capable of giving alarm to patient and nurses after receiving the alarm signal from the signal processor 30E. To remove the environmental interference, the two conductive wires 20U and 20V are made of coaxial cables while the outer shielding layers of the coaxial cables are connected to a reference point with zero potential, e.g., the negative pole of a battery 50. The assembly box 60 in FIG. 3A containing the signal processor 30E, the battery 50 and part of the alarm device 40B also provides shielding function.

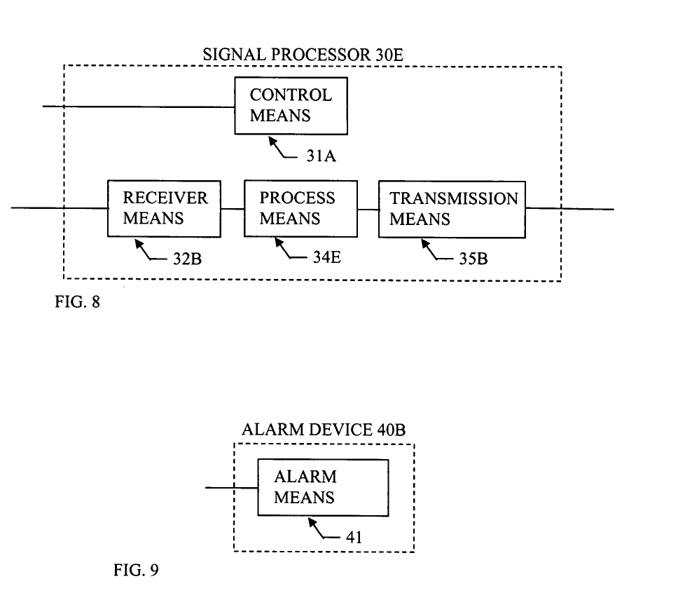

FIG. 8 is a block diagram of an exemplary signal processor 30E. the signal processor 30E comprises control means 31A for applying an electric current between the two conductive wires 20U and 20V in FIG. 7, receiver means 32B for receiving the electric parameters of the electric current, process means 34E for analyzing the received electric parameter and judging if the medical liquid 10 inside the IV bottle 11 has dropped below the predetermined low level, transmission means 35B for sending an alarm signal if the liquid level has dropped to the predetermined low level.

FIG. 9 is a block diagram of an exemplary alarm device 40B. The alarm device 40B includes alarm means 41 for giving alarm after receiving the alarm signal from the transmission means 35B in signal processor 30E of FIG. 8. The detailed alarm means is similar to that in the first embodiment described in FIG. 5A-5C.

What is claims is:

1. A portable IV infusion monitoring system, comprising:
(a) liquid level sensor including at least two electrodes outside an IV bottle for conducting an electric current between said two electrodes;
(b) a microprocessor having
control means for controlling said electric current between said two electrodes,
receiver means for receiving the electric signal of said electric current,
detector means for detecting the electric parameters of said electric signal,
process means for analyzing said electric parameters and obtaining the liquid level data inside said IV bottle,
transmission means for sending out said liquid level data;
(c) a monitor terminal having
alarm means responsive to said liquid level data for giving alarm.

2. The monitoring system of claim 1, wherein said monitoring system further comprising
a battery for providing an electric power to said monitoring system.

3. The monitoring system of claim 1, wherein said at least two electrodes being positioned at selected one of two sides in opposite direction and one side in parallel location of said IV bottle.

4. The monitoring system of claim 1, wherein said liquid level sensor further comprising at least two coaxial cables for connecting said at least two electrodes to said microprocessor, and the outer shielding layers of said at least two coaxial cables being connected to at least one of said battery, said microprocessor and a zero potential point.

5. The monitoring system of claim 1, wherein at least one shielding plate made of conductive materials being positioned at the outer surface of said at least two electrodes, and being connected to at least one of said battery, said microprocessor and a zero potential point.

6. The monitoring system of claim 1, wherein said monitoring system comprising an assembly box for containing said battery, said microprocessor, and part of said monitor terminal, and further having shielding means for shielding said microprocessor from environmental interference, said shielding means including selected one of said assembly box being made of metal and said assembly box being coated with conductive materials, said shielding means being connected to selected at least one of said battery, said microprocessor and a zero potential point.

7. The monitoring system of claim 1, wherein said liquid level sensor further comprising an electric bridge circuit for detecting said electric signal between said at least two electrodes.

8. The monitoring system of claim 1, wherein said microprocessor comprising
(a) receiver for receiving the electric signal of said electric current;
(b) detector for detecting the voltage signal from said electric signal;
(c) signal interface for storing said voltage signal;
(d) A/D converter for converting said voltage signal into digital data;
(e) digital register for storing said digital data;
(f) processor for analyzing said digital data and obtaining said liquid level data;
(g) output port for transmitting said liquid level data;
(h) program controller being programmed with unique software code for administrating the operation of all members from (a) to (g).

9. The monitoring system of claim 1, wherein said control means in said microprocessor further comprising means for controlling said electric current selected from the group including
(a) narrow band signal;
(b) multi frequency signal;
(c) encoded signal; and
said microprocessor further comprising interference filtering means for removing environmental interference, and said interference filtering means comprising a method selected from the group including
(a) narrow band filter for filtering out said narrow band signal;
(b) Fourier analyzer for picking up said multi frequency signal by Fourier analysis;
(c) decoder for decoding said encoded signal.

10. The monitoring system of claim 1, wherein said monitor terminal, comprising
at least one device selected from the group consisting of
(a) a sound generator responsive to said liquid level data for generating a loud sound when said liquid level inside said IV bottle drops to predetermined low level;
(b) a switch means responsive to said liquid level data for cutting off the feeding of the medical liquid within said IV bottle when said liquid level inside said IV bottle drops to said predetermined low level;
(c) a signal network responsive to said liquid level data for transmitting said liquid level data through signal network to a nurse station by a way selected from one of wire transmission and wireless transmission;
(d) display means for displaying said liquid level data.

11. The monitoring system of claim 1, wherein said monitor terminal, further comprising
a rate controller including
(a) an input port for inputting the desired infusion rate of the medical liquid inside said IV bottle;
(b) a comparator for comparing said desired infusion rate and the detected infusion rate;
(c) an electric switch means for adjusting the infusion rate according to the results from said comparator.

12. A portable IV infusion monitoring system, comprising:
(a) liquid level sensor including at least one electrode positioned outside an IV bottle, and one conductive wire for connecting selected one of a liquid needle and an air needle within said IV bottle;
(b) a microprocessor having
control means for controlling an electric current between said at least one electrode and said conductive wire,
receiver means for receiving the electric signal of said electric current,
detector means for detecting the electric parameters of said electric signal,
process means for analyzing said electric parameters and obtaining the liquid level data inside said IV bottle,
transmission means for sending out said liquid level data;
(c) a monitor terminal having
alarm means responsive to said liquid level data for giving alarm.

13. The monitoring system of claim 12, wherein said liquid level sensor further comprising an electric bridge circuit for detecting said electric signal between said at least one electrode and said conductive wire.

14. The monitoring system of claim 12, wherein said microprocessor comprising
(a) receiver for receiving the electric signal of said electric current;
(b) detector for detecting the voltage signal from said electric signal;
(c) signal interface for storing said voltage signal;
(d) A/D converter for converting said voltage signal into digital data;
(e) digital register for storing said digital data;
(f) processor for analyzing said digital data and obtaining said liquid level data;
(g) output port for transmitting said liquid level data;
(h) program controller programmed with unique software code for administrating the operation of all members from (a) to (g).

15. The monitoring system of claim 12, wherein said liquid level sensor further comprising first at least one coaxial cable for connecting said at least one electrode to said microprocessor, and said one conductive wire being made of second coaxial cable for connecting selected one of a liquid needle and an air needle to said microprocessor.

16. The monitoring system of claim 12, wherein said control means in said microprocessor further comprising means for controlling said electric current selected from the group including
(a) narrow band signal;
(b) multi frequency signal;
(c) encoded signal; and
said microprocessor further comprising interference filtering means for removing the interference from environment, and said interference filtering means comprising a method selected from the group including
(a) narrow band filter for filtering out said narrow band signal;
(b) Fourier analyzer for picking up said multi frequency signal by Fourier analysis;
(c) decoder for decoding said encoded signal.

17. The monitoring system of claim 12, wherein said monitor terminal further comprising at least one device selected from the group consisting of
(a) a sound generator responsive to said liquid level data for generating a loud sound when said liquid level inside said IV bottle drops to predetermined low level;
(b) a switch means responsive to said liquid level data for cutting off the feeding of the medical liquid within said IV bottle when said liquid level inside said IV bottle drops to said predetermined low level;
(c) a signal network responsive to said liquid level data for transmitting said liquid level data through signal network to a nurse station by a way selected from one of wire transmission and wireless transmission;
(d) display means for displaying said liquid level data.

18. A portable IV infusion monitoring system, comprising:
(a) liquid level sensor including a first conductive wire for connecting a liquid needle within an IV bottle, and a second conductive wire for connecting an air needle within said IV bottle;
(b) a signal processor, having
control means for controlling an electric current between said first conductive wire and said second conductive wire,
receiver means for receiving the electric signal of said electric current,
process means for analyzing the electric parameters of said electric signal and judging if the liquid level within said IV bottle drops to a predetermined low level,
transmission means for sending out an alarm signal when the medical liquid inside said IV bottle drops to said predetermined low level;
(c) an alarm device having
alarm means responsive to said alarm signal for giving alarm.

19. The monitoring system of claim 18, wherein said liquid level sensor further comprising
an electric bridge circuit for detecting said electric signal between said first conductive wire and said second conductive wire.

20. The monitoring system of claim 18, wherein said alarm device further comprising
at least one device selected from the group consisting of
(a) a sound generator responsive to said alarming signal for generating a loud sound;
(b) a switch means responsive to said alarming signal for cutting off the feeding of the medical liquid within said IV bottle;
(c) a signal network responsive to said alarm signal for transmitting said alarm signal through signal network to a nurse station by a way selected from one of wire transmission and wireless transmission;
(d) display means for displaying said alarm signal.
